(19) United States
(12) Patent Application Publication
(10) Pub. No.: US 2023/0364609 A1
(43) Pub. Date: Nov. 16, 2023

(54) SAMPLE PROCESSING DEVICE, SAMPLE PROCESSING APPARATUS, AND SAMPLE PROCESSING METHOD

(71) Applicant: Hitachi High-Tech Corporation, Tokyo (JP)

(72) Inventors: Yoshihiro NAGAOKA, Tokyo (JP); Shuhei YAMAMOTO, Tokyo (JP); Taro NAKAZAWA, Tokyo (JP)

(21) Appl. No.: 18/027,978

(22) PCT Filed: Oct. 6, 2020
(86) PCT No.: PCT/JP2020/037849

§ 371 Date: Mar. 23, 2023

(51) Int. Cl. B01L 3/00

(52) U.S. Cl. CPC B01L 3/50273; B01L 2200/141; B01L 2200/16; B01L 2300/0681; B01L 2400/0478; B01L 2300/18

(57) ABSTRACT

A sample processing device comprising a sample holding part, a reagent holding part, a reaction part, a flow path for connecting the sample holding part, reagent holding part, and reaction part, a plurality of cylinders, and a plurality of plungers respectively disposed in the plurality of cylinders so as to be capable of reciprocal motion, wherein fluid can circulate among the plurality of cylinders via the flow path and the cylinders are sealed by the plungers. This configuration makes it possible to maintain a sealed state such that an external substance does not enter into the sample processing device during sample processing and to easily carry out quantitative flow manipulation, and the like, of the sample.

### TECHNICAL FIELD

The present invention relates to a sample processing device, a sample processing apparatus, and a sample processing method.

### BACKGROUND ART

Conventionally, the microfluidic biochips that are capable of executing a series of complex processing steps in parallel on one or more samples in an integrated state without the need of operator intervention are under development.

WO2011/112746A (PTL 1) describes a single-structure biochip that provides processability from sample introduction to result output, in which pneumatic and vacuum pumps are connected to a pneumatic manifold through a series of tanks and solenoid valves, and the manifold is connected to the pneumatic port of the biochip via a pneumatic interface.

JP2014-18180A (PTL 2) describes a biochemical cartridge which is configured as a pump mechanism including a liquid-feeding source chamber in which a reagent to be fed is sealed, a liquid-feeding destination chamber of the reagent, and a liquid-feeding passage connecting the chambers, in which the chambers and liquid-feeding passage are provided in a sealed manner in the cartridge body, the liquid feed passage is formed on the bottom surface of the cartridge body, and a membrane made of an elastic material is attached, a part of the membrane becomes one of the wall surfaces of the liquid feeding passage, and the volume of the liquid-feeding passage is changed by reciprocating due to changes in the pressure applied from the outside. PTL 2 describes that the biochemical cartridge performs preprocessing from DNA extraction to amplification, sends the liquid processed in the biochemical cartridge to a capillary electrophoresis DNA sequencer, and performs DNA analysis.

### CITATION LIST

### Patent Literature

PTL 1: WO2011/112746A

PTL 2: JP2014-18180A

### SUMMARY OF INVENTION

### Technical Problem

The biochip described in PTL 1 uses air pressure to allow samples and reagents to flow within the biochip to perform a series of sample processing, but the pneumatic source is on the outside of the biochip and is connected to the pneumatic manifold, and accordingly, air is supplied to the biochip via the pneumatic interface. Therefore, when the pneumatic source is connected to the biochip, the pneumatic source and the biochip communicate with each other, and there is a possibility that substances moving in the biochip during a series of sample processing are moved to the pneumatic source side outside the biochip, and stick to a part that is in contact with, for example, the pneumatic manifold. When the biochip is removed after the process ends and another biochip is connected to the pneumatic source, there is a possibility that the substance previously stuck to the pneumatic source side will move into the biochip and contaminate the biochip.

The biochemical cartridge described in PTL 2 performs preprocessing up to DNA amplification, and DNA analysis is performed by a separate device. Therefore, there is room for improvement from the viewpoint of making the apparatus compact and completely preventing contamination of the sample. In the biochemical cartridge described in PTL 2, it is considered that the plunger having a pump function is provided outside the membrane, that is, outside the cartridge, and thus it is not easy to transport a large amount of liquid at high speed.

An object of the invention is to maintain a sealed state such that an external substance does not enter into the sample processing device during sample processing and to easily carry out quantitative flow manipulation, and the like, of the sample.

### Solution to Problem

In order to achieve the above object, there is provided a sample processing device of the invention including: a sample holding part; a reagent holding part; a reaction part; a flow path for connecting the sample holding part, the reagent holding part, and the reaction part; a plurality of cylinders; and a plurality of plungers respectively installed to be capable of reciprocating in the plurality of cylinders, in which the plurality of cylinders are configured to allow fluid to circulate through each other via the flow path, and the cylinder is sealed by the plunger.

There is provided a sample processing apparatus of the invention including: a drive part; a temperature adjusting part; a measuring part; and a stage on which the sample processing device is installable, in which the drive part has a plunger drive mechanism, and the plunger drive mechanism reciprocates the plurality of plungers.

### Advantageous Effects of Invention

According to the invention, it is possible to maintain a sealed state such that an external substance does not enter into the sample processing device during sample processing and to easily carry out quantitative flow manipulation, and the like, of the sample. The problems, configurations, and the effects of the invention other than those described above will be clarified from the description of the embodiments below.

### BRIEF DESCRIPTION OF DRAWINGS

FIG. 1A is a top view showing a sample processing device according to an example.

FIG. 1B is a side view showing the sample processing device.

FIG. 1C is a sectional view taken along line A-A of FIG. 1A.

FIG. 2A is a top view showing a main plate according to the example.

FIG. 2B is a sectional view taken along line B-B of FIG. 2A.

FIG. 3A is a top view showing a reagent vessel 41 of FIG. 1A.

FIG. 3B is a sectional view taken along line C-C of FIG. 3A.

FIG. 4 is a side view showing a sample processing apparatus according to the example.

FIG. 5A is a front view showing a connection state between a plunger drive mechanism and a plunger according to the example.

FIG. 5B is a side view of the state of FIG. 5A.

FIG. 6 is a flow diagram showing a sample processing method according to the example.

FIG. 7 is a flowchart showing details of a processing operation step S705 of FIG. 6.

FIG. 8A is a sectional view showing an internal state (initial state) of the sample processing device in the processing operation step S705 of FIG. 6.

FIG. 8B is a sectional view showing an internal state of the sample processing device in a reagent introduction step S711 of FIG. 7 (state where four types of reagents have been introduced).

FIG. 8C is a sectional view showing an internal state (state immediately after a flow) of the sample processing device in a sample flow step S712 of FIG. 7.

FIG. 8D is a sectional view showing an internal state (state immediately after a flow) of the sample processing device in a reagent flow step S713 of FIG. 7.

FIG. 8E is a sectional view showing an internal state (state during reaction) of the sample processing device in a reaction step S714 of FIG. 7.

FIG. 8F is a sectional view showing an internal state of the sample processing device in a collection step S715 of FIG. 7.

FIG. 8G is a sectional view showing an internal state (state after collection) of the sample processing device in the collection step S715 of FIG. 7.

FIG. 9A is a front view showing a modification example of the plunger drive mechanism and the plunger.

FIG. 9B is a side view of FIG. 9A.

FIG. 10 is a sectional view showing a sealing structure of an upper end portion of the cylinder.

### DESCRIPTION OF EMBODIMENTS

The invention relates to a sample processing device, a sample processing apparatus, and a sample processing method, and more particularly to a technology for performing a liquid flow manipulation within a sealed sample processing device.

The configuration and the like of the sample processing device of an example will be described below with reference to the drawings. In principle, the same number is attached to the same item in a plurality of drawings.

### EXAMPLE

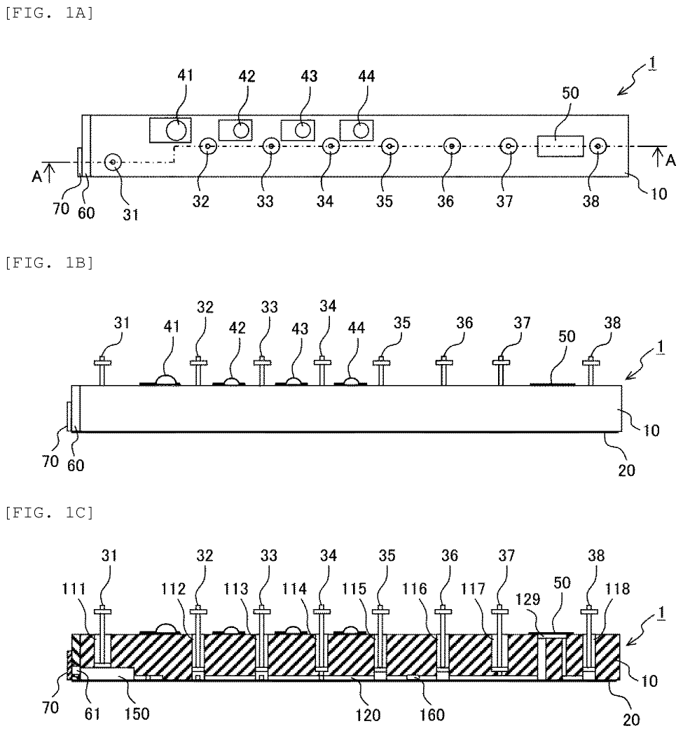

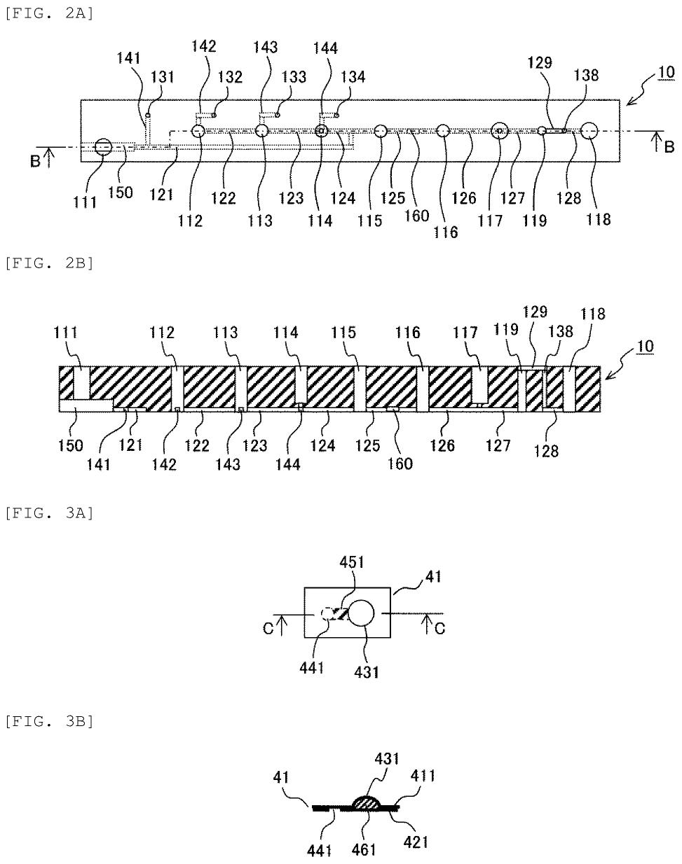

A sample processing device according to an example and a basic configuration of a sample processing apparatus including a stage on which the sample processing device can be installed will be described below with reference to FIGS. 1A to 5B.

The sample processing device of the present example has a configuration in which a liquid sample such as blood or urine, a liquid sample containing components eluted from a swab or the like, and a reagent can flow in a sealed state. The sample processing device performs substance identification, quantification, and the like.

FIG. 1A is a top view showing a sample processing device (cartridge) according to the present example.

As shown in the drawing, a plurality of plungers 31, 32, 33, 34, 35, 36, 37, and 38, a plurality of reagent vessels 41, 42, 43, and 44 (reagent holding parts), and an upper surface film 50 are provided in the upper surface portion of a main plate 10, which is the main portion of the sample processing device 1. A side plate 60 and a lid 70 are provided at one end portion (the left end in the drawing) of the main plate 10.

FIG. 1B is a side view showing a sample processing device according to the present example.

In the drawing, a state where the plungers 31, 32, 33, 34, 35, 36, 37, and 38 and the reagent vessels 41, 42, 43, and 44 protrude from the upper surface of the main plate 10 is shown. The lower surface of the main plate 10 is covered with a lower surface film 20.

FIG. 1C is a sectional view taken along line A-A of FIG. 1A.

As shown in FIG. 1C, the main plate 10 is provided with a plurality of cylinders 111, 112, 113, 114, 115, 116, 117, and 118.

A plurality of groove structures (recessed parts) are provided in the lower surface portion of the main plate 10. Since the groove structure is covered with the lower surface film 20, the groove structure constitutes a sample holding part 150, a flow path 120, and the like. A filter 160 is installed in the middle of the flow path 120. The filter 160 constitutes a reaction part for amplifying nucleic acids, although the details will be described later.

Each cylinder 111, 112, 113, 114, 115, 116, 117, and 118 communicates with the sample holding part 150, the flow path 120, and the like. The plungers 31, 32, 33, 34, 35, 36, 37, and 38 are mounted on cylinders 111, 112, 113, 114, 115, 116, 117, and 118, respectively. The length of the plungers 31, 32, 33, 34, 35, 36, 37, and 38 is greater than the depth of the cylinders 111, 112, 113, 114, 115, 116, 117, and 118. Therefore, the upper portion of the plungers 31, 32, 33, 34, 35, 36, 37, and 38 protrudes above the cylinders 111, 112, 113, 114, 115, 116, 117, and 118.

The plungers 31, 32, 33, 34, 35, 36, 37, and 38 can move up and down within the cylinders 111, 112, 113, 114, 115, 116, 117, and 118. Before mounting, the outer diameters of the plungers 31, 32, 33, 34, 35, 36, 37, and 38 are the same as the inner diameters of the cylinders 111, 112, 113, 114, 115, 116, 117, and 118, or are slightly greater than the inner diameters of the cylinders 111, 112, 113, 114, 115, 116, 117, and 118. As a result, the inner wall surfaces of the cylinders 111, 112, 113, 114, 115, 116, 117, and 118 and the outer peripheral surfaces of the plungers 31, 32, 33, 34, 35, 36, 37, and 38 are in close contact with each other. That is, the cylinders 111, 112, 113, 114, 115, 116, 117, and 118 are sealed on the upper surface side by the plungers 31, 32, 33, 34, 35, 36, 37, and 38, respectively.

A sample inlet 61 is provided on the side plate 60. The sample inlet 61 is sealed with a lid 70. The lower surface portion and the side surface portion of the sample holding part 150, the flow path 120, and the like are sealed by the lower surface film 20 and the lid 70.

The upper surface portion of the main plate 10 is also provided with an upper surface flow path 129 having a groove structure. The upper surface flow path 129 is covered and sealed with the upper surface film 50.

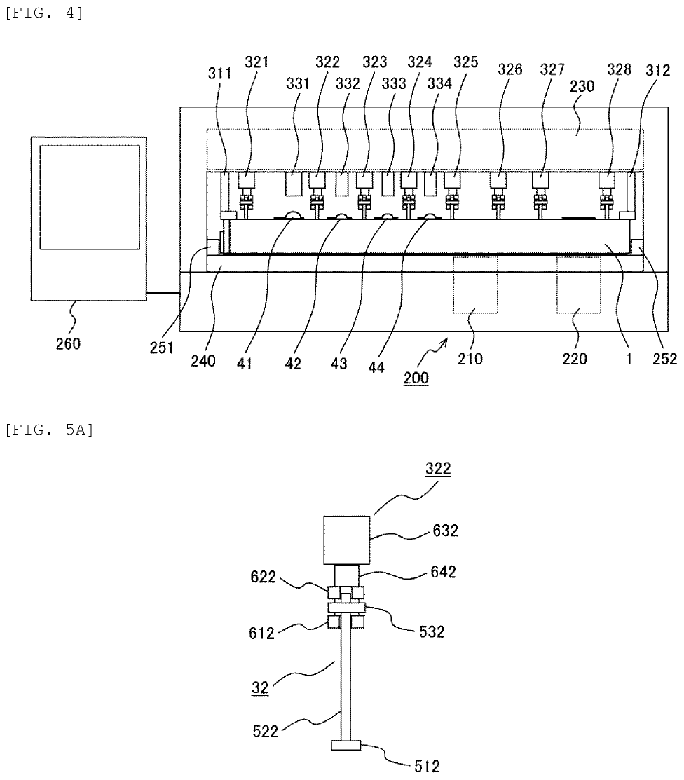

A plurality of reagent vessels 41, 42, 43, and 44 are installed in the upper surface portion of the main plate 10. The groove structure provided in the lower surface portion of the main plate 10 partially communicates with the groove structure on the upper surface portion of the main plate 10, but is sealed by the reagent vessels 41, 42, 43, and 44. Details of the reagent vessels 41, 42, 43, and 44 will be described later with reference to FIGS. 2A to 3B.

With the above configuration, the cylinders 111, 112, 113, 114, 115, 116, 117, and 118, the sample holding part 150, the flow path 120, and the like are sealed as a whole. That is, the inside of the sample processing device 1 is isolated from the outside.

The dimensions of the sample processing device of the present example are approximately 130 mm in length, 18 mm in width, and 5 mm in thickness (height).

FIG. 2A is a top view showing the main plate according to the example.

FIG. 2B is a sectional view taken along line B-B of FIG. 2A. In FIG. 2A, the groove structure on the lower surface side is indicated by broken lines. It should be noted that in the following description, the description that overlaps with the description of FIGS. 1A to 1C will be omitted.

As shown in FIG. 2B, the cylinders 112, 113, 114, 115, 116, and 117 communicate with each other by flow paths 122, 123, 124, 125, and 126, respectively. The cylinder 111 communicates with the sample holding part 150.

As shown in FIG. 2A, the sample holding part 150 communicates with the flow path 124 via the flow path 121. The cylinder 117 and the cylinder 118 communicate with each other via a flow path 127, a collected liquid holding part 119, an upper surface flow path 129, a communication flow path 138, and a flow path 128.

In summary, the cylinders 111, 112, 113, 114, 115, 116, and 117 are configured to allow the fluid to circulate through each other via flow paths 121, 122, 123, 124, 125, 126, and the like.

As shown in FIG. 2B, the collected liquid holding part 119 and the communication flow path 138 are through-holes formed in the vertical direction, similar to the cylinders 112, 113, 114, 115, 116, and 117. Therefore, the flow path 127 provided in the lower surface portion of the main plate 10 communicates with the upper surface flow path 129 provided in the upper surface portion of the main plate 10 via the collected liquid holding part 119, and communicates with the flow path 128 provided in the lower surface portion of the main plate 10 via the communication flow path 138.

The filter 160 is provided in the flow path 125.

As shown in FIG. 2A, the main plate 10 is provided with reagent introduction holes 131, 132, 133, and 134, which respectively communicate with the flow path 121, the cylinder 112, the cylinder 113, and the cylinder 114 via reagent introduction flow paths 141, 142, 143, and 144.

FIG. 3A is a top view showing the reagent vessel 41 of FIG. 1A.

FIG. 3B is a sectional view taken along line C-C of FIG. 3A.

As shown in FIG. 3B, the reagent vessel 41 includes a reagent upper film 411 and a reagent lower surface film 421. The reagent upper film 411 includes a protruding part, and a reagent storage part 431 is provided between the protruding part and the reagent lower surface film 421. A reagent 461 is held in the reagent storage part 431. The reagent lower surface film 421 is provided with a film removing part 441 in which the film is removed in a circular shape. While the reagent 461 is held, except for the reagent storage part 431 and the film removing part 441, the reagent upper film 411 and the reagent lower surface film 421 have contact surfaces, and the contact surfaces are joined to form a joint.

A low-strength joint 451 hatched in FIG. 3A has weaker joining strength than other joints. Therefore, there is no outflow during transportation or storage, but by an operation such as crushing the protruding part of the reagent upper film 411 from above, only the low-strength joint 451 is peeled off, the reagent storage part 431 and the film removing part 441 communicate with each other, and the reagent can flow out from the film removing part 441.

Other reagent vessels 42, 43, and 44 have similar structures.

Each of the reagent vessels 41, 42, 43, and 44 is joined to the upper surface portion of the main plate 10 (FIG. 1B) such that the film removing part is aligned with the reagent introduction hole, for example, the film removing part 441 and the reagent introduction hole 131 are aligned with each other. Therefore, the reagent flowing out from the film removing part flows into the reagent introduction hole.

The reagent is sealed in the reagent vessel, and when the reagent storage part, which is a protruding part, is crushed, the low-strength joint is peeled off, and the reagent storage part and the reagent introduction hole communicate with each other, but without communication with the outside, and the sample processing device is sealed.

FIG. 4 is a side view showing a sample processing apparatus according to the present example.

As shown in the drawing, a sample processing apparatus 200 includes a temperature control part 210 (temperature adjusting part), a measuring part 220, a drive part 230, and a stage 240.

The temperature control part 210 adjusts the temperature of the mixed liquid and the filter in the flow path of the sample processing device 1 installed on the stage 240 by heating or cooling. As a heating means, an electric heater, a heat pump, a Peltier element, or the like can be used. As the cooling means, air cooling, water cooling, heat pump, heat pipe, Peltier element, and the like can be used. Note that the temperature control part 210 may be configured to adjust the temperature of at least one of the sample holding part, the reagent holding part, the reaction part, and the collected liquid holding part by heating or cooling. It is because it becomes possible to preheat before the reaction in the reaction part. It is also possible to adjust the temperature when the reaction is caused in the liquid other than the reaction part.

The measuring part 220 includes an optical device such as an absorbance detector and a fluorescence detector, and performs optical measurements such as irradiating a mixed liquid or the like with light, detecting transmitted light, scattered light, or fluorescence from the mixed liquid or the like.

The drive part 230 includes a plurality of motors. These motors are drive sources for plunger drive mechanisms 321, 322, 323, 324, 325, 326, 327, and 328, device fixing mechanisms 311 and 312, and reagent introduction mechanisms 331, 332, 333, and 334, respectively. Rotational motion of the motor of the drive part 230 is converted into up-down operation.

The sample processing device 1 is installed on the stage 240.

An auxiliary device 260 is also connected to the sample processing apparatus 200. The auxiliary device 260 controls various operations including start-up and termination, sets processing conditions, records the operation status, displays the results, and the like for the sample processing apparatus 200. The auxiliary device 260 may be built in the sample processing apparatus 200.

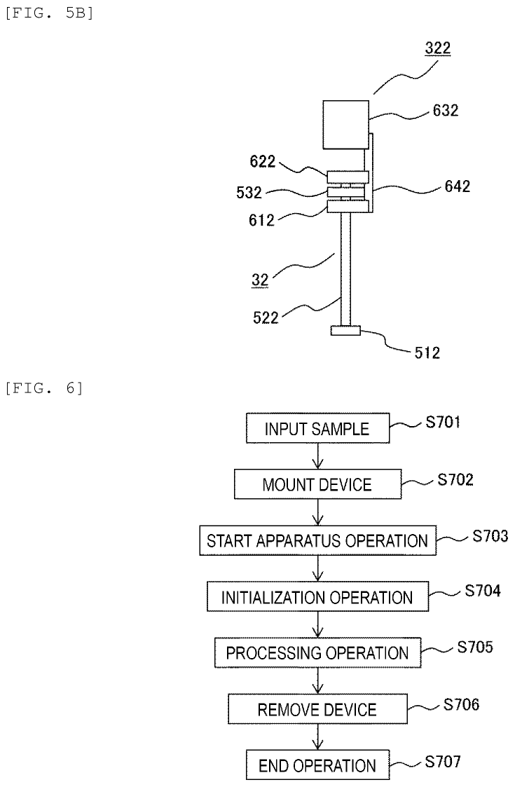

The sample processing device 1 is inserted all the way in the perpendicular direction in the drawing such that the upper surface of the stage 240 is slid. Here, the guides 251 and 252 at both end portions of the stage 240 determine the position of the sample processing device 1, and each of the plungers 31, 32, 33, 34, 35, 36, 37, and 38 (FIG. 1B) is connected to the plunger drive mechanisms 321, 322, 323, 324, 325, 326, 327, and 328 (details will be described later with reference to FIGS. 5A and 5B).

The plunger drive mechanisms 321, 322, 323, 324, 325, 326, 327, and 328 are coupled to the sample processing device 1 on the outside of the cylinder as each of the plungers 31, 32, 33, 34, 35, 36, 37, and 38 (FIG. 1B) protrudes above the sample processing device 1.

When the sample processing device 1 is inserted, the device fixing mechanisms 311 and 312 are lowered to press the sample processing device 1 against the stage 240 and fix the sample processing device 1. The reagent introduction mechanisms 331, 332, 333, and 334 are arranged to be positioned immediately above the reagent vessels 41, 42, 43, and 44, respectively. The reagent introduction mechanisms 331, 332, 333, and 334 are lowered according to control signals from the auxiliary device 260, respectively, and crush protruding parts of each of the reagent vessels 41, 42, 43, and 44, thereby introducing the reagent into a predetermined flow path or the like of the sample processing device 1.

FIG. 5A is a front view showing a connection state between a plunger drive mechanism and a plunger according to the present example.

FIG. 5B is a side view of the state of FIG. 5A. As an example, the plunger 32 and the plunger drive mechanism 322 will be described.

As shown in FIG. 5A, the plunger 32 has a sealing tip 512 that is movable in the cylinder and seals the cylinder at the lower end portion, has a disk-shaped projected part 532 in the upper portion thereof, and has a plunger shaft 522 that connects with the sealing tip 512 and the projected part 532.

The plunger drive mechanism 322 includes a lower surface holding part 612, an upper surface holding part 622, a motor connecting part 632, and a coupling part 642. The lower surface holding part 612 and the upper surface holding part 622 are configured to sandwich the projected part 532 of the plunger 32 up and down. The lower surface holding part 612, the upper surface holding part 622, and the motor connecting part 632 are coupled by the coupling part 642.

As shown in FIG. 5B, the projected part 532 is sandwiched between the lower surface holding part 612 and the upper surface holding part 622 from behind. In other words, the plunger drive mechanism 322 is coupled to the plunger 32 via the projected part 532.

As a result, the plunger drive mechanism 322 moves up and down according to the operation of the motor, and power is transmitted to the projected part 532 sandwiched between the lower surface holding part 612 and the upper surface holding part 622. Alongside, the plunger 32 moves up and down.

In summary, a plurality of plungers are installed to be capable of reciprocating on each of the plurality of cylinders.

Next, the operation of the sample processing device and the sample processing apparatus of the present example will be described.

FIG. 6 is a flow diagram showing a sample processing method according to the present example.

As shown in the drawing, in a sample input step S701, the operator opens the lid 70 (FIG. 1C), inputs the sample from the sample inlet 61 into the sample holding part 150, closes the lid 70, and seals the sample processing device 1.

In the next device mounting step S702, the sample processing device 1 is placed on the stage 240 of the sample processing apparatus 200 (FIG. 4) and inserted all the way to slide along the guides 251 and 252.

In the next apparatus operation start step S703, the operator selects an item according to the content of analysis using the auxiliary device 260 (FIG. 4) and starts an apparatus operation.

Next, the sample processing apparatus 200 starts an initialization operation step S704, lowers the device fixing mechanisms 311 and 312, and presses the sample processing device 1 against the stage 240 to fix the sample processing device 1. Preparation operations for mechanical systems such as the plunger drive mechanism and the reagent introduction mechanism, and checking of the temperature control part and measuring part are performed.

In the next processing operation step S705, a series of sample processing is performed in the sample processing device 1, and the processing results are stored in the memory within the sample processing apparatus 200, and displayed on the display of the auxiliary device 260, and the like, as required.

When the processing operation step S705 is finished, the operator removes the sample processing device 1 for storage and disposal in a device removal step S706.

When there is a next sample to be processed, the process returns to the sample input step S701, the sample is input into the new sample processing device 1, the sample processing device 1 is installed in the sample processing apparatus 200 (step S702), and the above sample processing (steps S703 to S706) are performed. When there is no new processing, the operator performs an end operation step S707 to stop the apparatus.

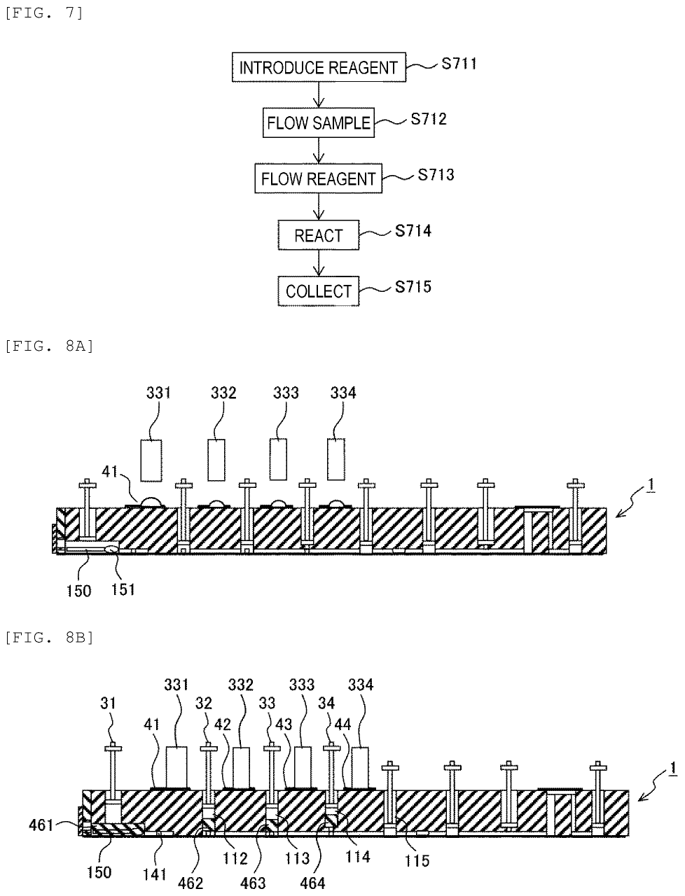

FIG. 7 is a flowchart showing details of the processing operation step S705 of FIG. 6.

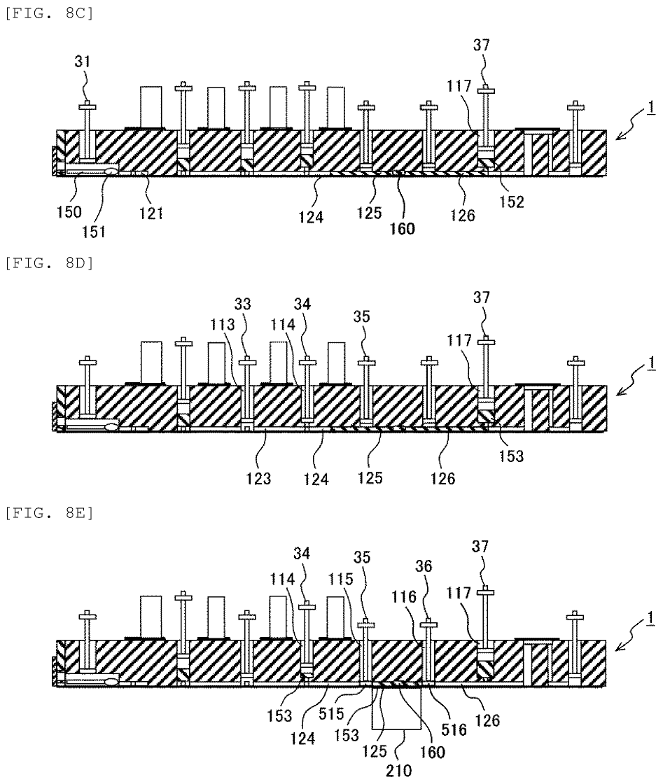

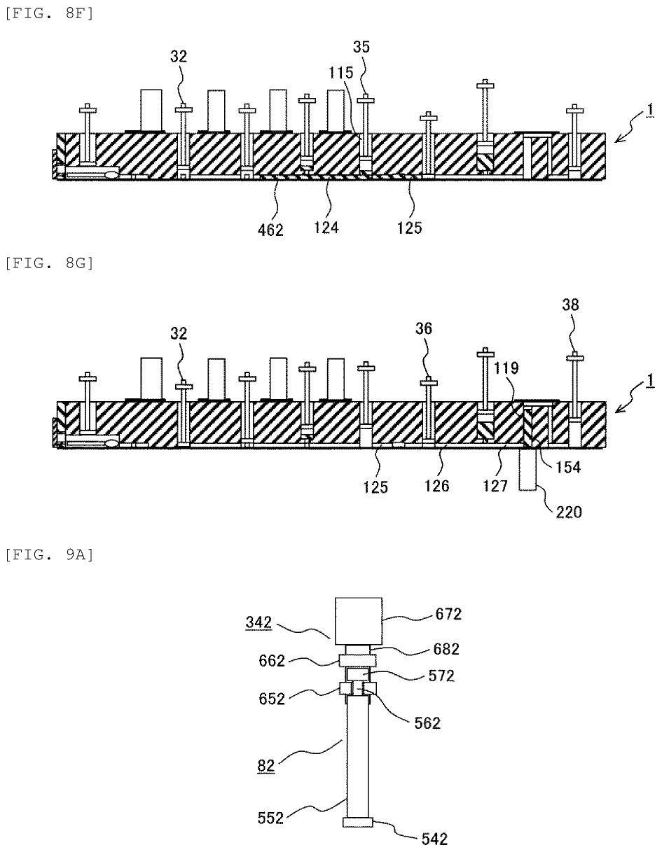

FIGS. 8A to 8G show changes in the state inside the sample processing device (section A-A in FIG. 1A) in the processing operation step S705. In the present examples, a processing operation is described in which intracellular cells are swabbed and intracellular nucleic acids are amplified in the cartridge.

FIGS. 8A to 8G show four reagent introduction mechanisms 331, 332, 333, and 334 in addition to the sample processing device 1.

FIG. 8A shows the initial state.

In the drawing, a swab 151 is input to the sample holding part 150. The reagent introduction mechanisms 331, 332, 333, and 334 of the sample processing device 1 (FIG. 4) are positioned immediately above the reagent vessels 41, 42, 43, and 44, respectively.

The first operation is the reagent introduction step S711 (FIG. 7), and four types of reagents are introduced in the present example.

FIG. 8B shows a state where four types of reagents 461, 462, 463, and 464 have been introduced. Here, the reagents 461, 462, 463, and 464 are introduced one by one.

For the reagent 461, for example, when the reagent introduction mechanism 331 is first lowered to crush the reagent vessel 41, the reagent 461 flows from the film removing part 441 (FIG. 3) into the reagent introduction hole 131 (FIG. 2A). When the plunger 31 is raised simultaneously, the reagent 461 flows into the sample holding part 150 through the reagent introduction flow path 141 and the flow path 121.

For the other reagents 462, 463, and 464, similarly, the reagent introduction mechanisms 332, 333, and 334 are lowered to crush the reagent vessels 42, 43, and 44, respectively, the plungers 32, 33, and 34 are raised simultaneously, and then each reagent flows into cylinders 112, 113, and 114. Here, to keep the pressure in the sample processing device 1 constant, it is desirable to move the plunger such that the product of the cylinder cross-sectional area and the movement amount, which is the amount of volume change due to the movement of each plunger, is approximately equal to the internal volume of the reagent vessel, which is the amount of volume change due to the crushing of the reagent vessel. Alternatively, the amount of volume change due to the lowering may be controlled to always be equal to or greater than the amount of volume change due to the raising. That is, the pressure in the sample processing device 1 is controlled to be lower than or equal to the pressure in the plunger stop state.

When the pressure is controlled as such, even if the flow path is branched, even when the liquid once flows into the flow path on the branch side, the liquid returns when the plunger stops, and thus the quantitative performance is not impaired.

Note that the introduction of the reagent does not need to be performed first, and may be performed immediately before the reagent is used.

The next operation is the sample flow step S712 (FIG. 7), and the state immediately after the flow is shown in FIG. 8C.

Specifically, by lowering the plunger 31 and raising the plunger 37, the sample 152 is caused to flow from the sample holding part 150 into the flow path 121, and flow into the cylinder 117 through the flow paths 124, 125, and 126. That is, by interlocking the plunger 31 on the upstream side (lowering) and the plunger 37 on the downstream side (raising), the sample 152 is allowed to flow in the flow path coupling both of the plungers. Here, to keep the pressure in the sample processing device 1 constant, it is desirable to move the plungers 31 and 37 such that the amount of volume change due to the movement of both of the plungers 31 and 37 is approximately equal.

The sample 152 is a liquid in which the reagent 461 is flowed into the sample holding part 150 and the nucleic acid, which is the substance to be processed, is eluted from the swab 151 into the reagent 461. Nucleic acids are captured by the filter 160 when the sample 152 passes through the filter 160 in the flow path 125. The fluid moved to the filter 160 (reaction part) is sealed by plungers on the upstream and downstream sides of the filter 160 among the plurality of plungers.

The next operation is a reagent flow step S713 (FIG. 7), and the state immediately after the flow is shown in FIG. 8D.

Specifically, by lowering the two plungers 33 and 34 and raising the plunger 37, the two types of reagents 463 and 464 are caused to flow from the cylinders 113 and 114 into the cylinder 117. However, first, the plunger 33 is lowered to allow the reagent 463 to flow into the flow path 123 to fill the flow path 123, and then the two plungers 33 and 34 are lowered simultaneously, such that the two types of reagents 463 and 464 are introduced into the flow path 124 simultaneously. The reagent 463 and the reagent 464 are mixed to form a mixed liquid 153, which passes through the filter 160 in the flow path 125 and flows into the cylinder 117 via the flow path 126. That is, by interlocking two plungers 33 and 34 on the upstream side (lowering) and the plunger 37 on the downstream side (raising), the reagent and the mixed liquid is allowed to flow in the flow path coupling both of the plungers.

When the two plungers on the upstream side perform the lowering operation simultaneously as such, to keep the pressure in the sample processing device 1 constant, it is desirable to move the plungers such that the total amount of volume change due to movement of the two plungers on the upstream side that performs the lowering operation is approximately equal to the amount of volume change due to movement of the plunger on the downstream side that performs the raising operation.

Even when the mixed liquid 153 is passed through the filter 160, the nucleic acids are not eluted from the filter 160, and are held in the mixed liquid together with the filter 160. The two types of reagents are an enzyme-mixed reagent and a primer-mixed reagent for amplifying nucleic acids, and the nucleic acids are amplified by controlling the temperature of the flow path 125 in subsequent steps. Therefore, the mixed liquid 153 only needs to be stopped to fill the flow path 125 after flowing, and does not necessarily need to flow into the cylinder 117.

The next operation is the reaction step S714 (FIG. 7), which is shown during the reaction in FIG. 8E.

Specifically, by lowering the plunger 35 and raising the plunger 34, the mixed liquid 153 is allowed to flow into the cylinder 114 from the flow path 124. By lowering the plunger 36 and raising the plunger 37, the mixed liquid 153 is allowed to flow into the cylinder 117 from the flow path 126. Here, the two lowered plungers 35 and 36 are lowered to the lower ends of the cylinders 115 and 116, and sealing tips 515 and 516 seal both ends of the flow path 125. Here, the temperature control part 210 controls the temperature of the mixed liquid in the flow path 125 and the temperature of the filter 160 to amplify the nucleic acid captured by the filter 160.

The next operation is the collection step S715, which shows the operation states in FIGS. 8F and 8G.

First, in FIG. 8F, the plunger 35 is raised and the plunger 32 is lowered to allow the flow path 125 to communicate with the flow path 124 and allow the reagent 462 to flow into the flow path 124. Here, the amount of movement of both plungers is adjusted such that the reagent 462 that has flowed into the flow path 124 comes into contact with the mixed liquid 153 in the flow path 125. When air enters between the reagent 462 and the mixed liquid 153, subsequent measurements are affected, and thus, a small amount of the reagent 462 may be flowed into the cylinder 115 as shown in FIG. 8F.

Next, as shown in FIG. 8G, the operation of allowing a collected liquid 154 in which the reagent 462 and the mixed liquid 153 are mixed to flow into the collected liquid holding part 119 will be described.

First, the plunger 36 is raised to allow the flow path 125 to communicate with the flow path 126. Here, the plunger 38 is lowered slightly such that the volume change is the same as the volume change due to the movement of the plunger 36.

By lowering the plunger 32 and raising the plunger 38, the reagent 462 and the mixed liquid 153 are allowed to flow into the flow path 126 side. As a result, the two liquids pass through the flow path 127 while being mixed, and flow into the collected liquid holding part 119 as the collected liquid 154. The collected liquid 154 contains eluted nucleic acids amplified in the filter 160.

The collected liquid 154 in the collected liquid holding part 119 is irradiated with excitation light by an optical device provided in the measuring part 220, and optical measurement such as fluorescence strength measurement is performed. Alternatively, measurement may be performed using another analysis device such as inserting a glass capillary into the collected liquid holding part 119 to perform electrophoresis. That is, the measuring part 220 may have an electrophoresis part or the like. In the case of electrophoresis, it is desirable to provide a small hole for connection to a glass capillary in the sample processing device 1 in advance, seal the small hole by covering the small hole with a film, and to connect the glass capillary such that the liquid inside the sample processing device 1 is not contaminated. The DNA sequencer may be included in the measuring part 220 of the sample processing apparatus 200 (FIG. 4).

In the present specification, optical measurement, electrophoresis, and processing by a DNA sequencer are collectively referred to as an “analysis step”.

The sample processing device 1 may have an integrated structure including a glass capillary and an electrode for electrophoresis. With such a configuration, a voltage can be applied to the electrodes from the outside, and processing by electrophoresis can be easily performed. As a result, contamination of the liquid inside the sample processing device 1 can be prevented in all the steps of reagent introduction, sample flow, collection, and analysis after sample input.

As described above, the flow manipulation inside the sample processing device is due to the operation of the plunger inside the sample processing device, and there is no possibility that equipment outside the sample processing device communicates with the flow path or the like inside the device. All connections of the mechanical system for operating the plunger are configured not to be in contact with the inside of the sample processing device, and as shown in FIGS. 5A and 5B, the plunger drive mechanism is connected to the upper end side of the plunger and does not enter the cylinder.

In the connection method of FIGS. 5A and 5B, the projected part on the upper end side of the plunger is used, but a recessed part may be provided for connection.

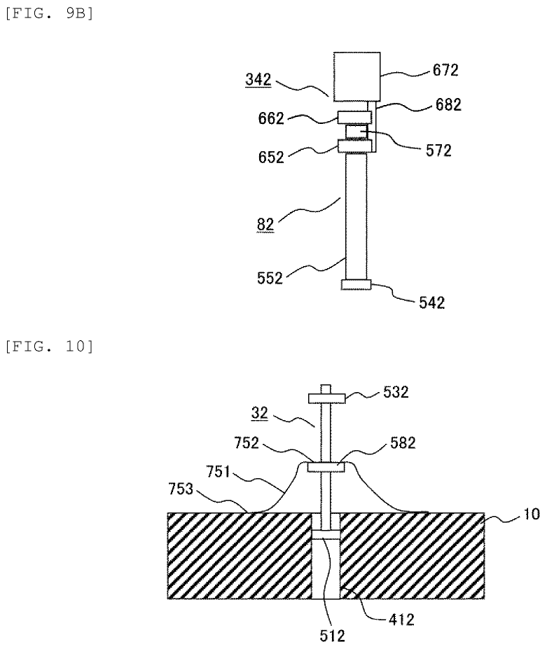

FIG. 9A is a front view showing a connection state of a modification example of the plunger drive mechanism and the plunger.

FIG. 9B is a side view of the state of FIG. 9A.

As shown in FIG. 9A, the plunger 82 is provided with a recessed part 562 in the middle, and the lower part of the recessed part 562 is a lower shaft portion 552 and the upper part of the recessed part 562 is an upper shaft portion 572. In other words, the recessed part 562 is provided between the lower shaft portion 552 and the upper shaft portion 572. A sealing tip 542 is attached to the lower end portion of the lower shaft portion 552 to move and seal the inside of the cylinder.

A plunger drive mechanism 342 includes a lower holding part 652 that is inserted into the recessed part 562 of the plunger 82, an upper holding part 662 that comes into contact with the upper end portion of the plunger 82, a coupling part 682, and a motor connecting part 672. In other words, the plunger drive mechanism 342 is coupled to the plunger 82 via the recessed part 562.

As shown in FIG. 9B, the lower holding part 652 and the upper holding part 662 are coupled by the coupling part 682. The coupling part 682 is connected to the motor connecting part 672. The lower holding part 652 pushes up the upper shaft portion 572 when the plunger 82 is raised, and the upper holding part 653 pushes down the upper shaft portion 572 when the plunger 82 is lowered. Alternatively, when the plunger 82 is lowered, a structure in which the lower holding part 652 pushes down the lower shaft portion 552 may be employed. Here, the upper shaft portion 572 is not needed to be provided.

In the present example, the plunger seals the cylinder, but when the plunger is lowered, the upper portion of the cylinder is exposed to the outside (atmosphere) of the sealing tip.

Therefore, in order to avoid such exposure, it is desirable to seal the end portion of the cylinder with an easily deformable film material.

FIG. 10 is a sectional view showing the sealing structure of the upper end portion of the cylinder according to the present example.

In the drawing, a joining plate 582 is provided on the shaft portion between the sealing tip 512 and the projected part 532 of the plunger 32. The plunger 32 penetrates a center portion 752 of the sealing film 751 and is joined to the sealing film 751 in a state where the joining plate 582 is on the main plate 10 side. The outer peripheral end portion 753 of the sealing film 751 is joined to the upper surface portion of the main plate 10 along the entire periphery. As a result, the upper end side of the cylinder 412 is sealed. A part of the plunger 32 is arranged below the sealing film 751 that seals the upper end side of the cylinder 412. By providing the sealing film 751, it is possible to more reliably prevent other substances from being mixed into the sample or the like.

Although the plunger 32 shown in the drawing is configured to penetrate the sealing film 751, the sealing film 751 may be configured to cover the upper end portion of the plunger 32, in other words, the entire plunger 32 may be in a state of being on the main plate 10 side. Here, it is desirable to have a configuration in which the upper end surface of the plunger 32 is provided with a recessed part, the protruding part provided at the lower end portion of the motor connecting part can be fitted into the recessed part of the plunger 32 and fixed by hooking the protruding part inside the recessed part. Accordingly, the entire plunger 32 is arranged below the sealing film 751 that seals the upper end side of the cylinder 412.

As described above, it is desirable for at least a part of the plunger 32 to be arranged below the sealing film 751 that seals the upper end side of the cylinder 412.

According to the present example, by lowering the plunger on the upstream side and raising the plunger on the downstream side, it is possible to feed the liquid only to the flow path that allows both of the plungers to communicate with each other. Therefore, there is no need for a valve mechanism for changing the liquid feeding path.

According to the present example, liquids such as samples and reagents are transported by raising and lowering the plunger inserted in the cylinder, and thus quantitative processing can be reliably performed. Since the movement distance of the plunger corresponds to the depth of the cylinder, a relatively large amount of liquid stored in the cylinder provided according to the volume of the reagent or the like to be used can be easily transported at high speed. Since the volume of the cylinder set by the diameter and depth of the cylinder can be designed in any manner, the stroke (reciprocating distance) of the plunger can be increased, and the amount and speed of liquid transported by the plunger can be easily controlled.

From the viewpoint of adjusting the moving distance and speed of the plunger, it is desirable to use a stepping motor as the motor of the drive part of the sample processing apparatus. Alternatively, a method in which one pneumatic source may be used to drive a plurality of plungers may be employed. A configuration using a pneumatic source is advantageous in terms of cost.

According to the present example, since the sample processing device performs sample processing in a sealed state, there is no movement of substances between the inside and the outside of the sample processing device, and it is possible to prevent environmental contamination due to leakage of substances generated in the sample processing device to the outside, and erroneous processing due to mixing or the like of another sample into the sample processing device.

### REFERENCE SIGNS LIST

1: Sample processing device
10: Main plate
20: Lower surface film
31, 32, 33, 34, 35, 36, 37, 38: Plunger
41, 42, 43, 44: Reagent vessel
50: Upper surface film
60: Side plate
61: Sample inlet
70: Lid
111, 112, 113, 114, 115, 116, 117, 118: Cylinder
119: Collected liquid holding part
120, 121, 122, 123, 124, 125, 126, 127, 128: Flow path
129: Upper surface flow path
131, 132, 133, 134: Reagent introduction hole
138: Communication flow path
141, 142, 143, 144: Reagent introduction flow path
150: Sample holding part
160: Filter
200: Sample processing apparatus
210: Temperature control part
220: Measuring part
230: Drive part
240: Stage
251, 252: Guide
260: Auxiliary device
311, 312: Device fixing mechanism
321, 322, 323, 324, 325, 326, 327, 328: Plunger drive
mechanism
331, 332, 333, 334: Reagent introduction mechanism
411: Reagent upper film
421: Reagent lower surface film
431: Reagent storage part
441: Film removing part
451: Low-strength joint
461: Reagent
512: Sealing tip
522: Plunger shaft
532: Projected part
612: Lower surface holding part
622: Upper surface holding part
632: Motor connecting part
642: Coupling part
751: Sealing film

1. A sample processing device comprising:
a sample holding part;
a reagent holding part;
a reaction part;
a flow path for connecting the sample holding part, the reagent holding part, and the reaction part;
a plurality of cylinders; and
a plurality of plungers respectively installed to be capable of reciprocating in the plurality of cylinders, wherein
the plurality of cylinders have a configuration that allows fluid to circulate through each other via the flow path, and
the cylinder is sealed by the plunger.

2. The sample processing device according to claim 1, wherein
the plunger has a projected part or a recessed part at a part positioned outside the cylinder, and is driven by a plunger drive mechanism coupled via the projected part or the recessed part.

3. The sample processing device according to claim 2, wherein
the plunger is longer than a depth of the cylinder.

4. The sample processing device according to claim 1, wherein
at least a part of the plunger is disposed below a sealing film that seals an upper end side of the cylinder.

5. A sample processing apparatus comprising:
a drive part;
a measuring part; and
a stage on which the sample processing device according to claim 1 is installable, wherein
the drive part has a plunger drive mechanism, and
the plunger drive mechanism reciprocates the plurality of plungers.

6. The sample processing apparatus according to claim 5, wherein
the plunger drive mechanism is coupled with the sample processing device on the outside of the cylinder.

7. The sample processing apparatus according to claim 5, wherein
the fluid moved to the reaction part is sealed by plungers on upstream and downstream sides of the reaction part among the plurality of plungers.

8. The sample processing apparatus according to claim 5, further comprising:
a temperature adjusting part, wherein
the temperature adjusting part adjusts, by heating or cooling, a temperature of at least one of the sample holding part, the reagent holding part, and the reaction part of the sample processing device installed on the stage.

9. The sample processing apparatus according to claim 5, wherein
the measuring part has a configuration capable of optical measurement.

10. The sample processing apparatus according to claim 5, wherein
the measuring part includes an electrophoresis part.

11. The sample processing apparatus according to claim 5, wherein
the plunger drive mechanism allows the fluid to circulate by reciprocating the plunger on the upstream side and the plunger on the downstream side included in the plurality of plungers.

12. The sample processing apparatus according to claim 5, wherein
the drive part has a reagent introduction mechanism, and
the reagent introduction mechanism is lowered to crush the reagent holding part.

13. A sample processing method which is a method for processing a sample using the sample processing apparatus according to claim 5, the method comprising:
a reagent introduction step of introducing a reagent into a predetermined position;
a sample flow step of flowing the sample in the sample holding part into a predetermined position;
a reaction step of allowing the reagent to react with a substance to be processed contained in the sample at a predetermined position; and
an analysis step of analyzing the fluid, wherein
the sample flow step is performed by reciprocating the plunger.

14. The sample processing method according to claim 13, wherein
the sample processing apparatus further includes a temperature adjusting part, and
the temperature adjusting part adjusts, by heating or cooling, a temperature of at least one of the sample holding part, the reagent holding part, and the reaction part of the sample processing device installed on the stage.

15. The sample processing method according to claim 13, wherein
the measuring part has a configuration capable of optical measurement, and
the analysis step is to perform the optical measurement.

16. The sample processing method according to claim 13, wherein
the measuring part includes an electrophoresis part, and the analysis step is performed by the electrophoresis part.

17. The sample processing method according to claim 13, wherein
the plunger drive mechanism allows the fluid to circulate by reciprocating the plunger on the upstream side and the plunger on the downstream side included in the plurality of plungers.

18. The sample processing method according to claim 13, wherein
the drive part has a reagent introduction mechanism, and
the reagent introduction step includes a step in which the reagent introduction mechanism is lowered to crush the reagent holding part.
